(19) United States
(12) Patent Application Publication
(10) Pub. No.: US 2013/0336899 A1
(43) Pub. Date: Dec. 19, 2013

(54) ANTIMICROBIAL FORMULATIONS THAT AID IN WOUND HEALING

(71) Applicants: Jianming Li, Lafayette, IN (US); Sean Connell, West Lafayette, IN (US)

(72) Inventors: Jianming Li, Lafayette, IN (US); Sean Connell, West Lafayette, IN (US)

(73) Assignee: Purdue Research Foundation, West Lafayette, IN (US)

(21) Appl. No.: 13/878,932

(22) PCT Filed: Oct. 11, 2011
(86) PCT No.: PCT/US2011/055810

§ 371 Date: Jul. 1, 2013

Related U.S. Application Data
(60) Provisional application No. 61/391,710, filed on Oct. 11, 2010.

(51) Int. Cl. A61K 31/734; A61K 31/14; A61K 31/722; A61K 31/7016; A61K 45/06; A61K 31/05

(52) U.S. Cl. CPC A61K 31/734; A61K 45/06; A61K 31/05; A61K 31/722; A61K 31/7016; A61K 31/14
USPC 424/10.3; 424/618; 424/702; 514/54; 514/55; 514/53

(57) ABSTRACT

The invention discloses compositions and formulations for the treatment of clean or infected wounds, some of the inventive formulations can be used to reduce or eliminate microbial contamination from surfaces such as skin, and inanimate objects such as countertops, cooking utensils, medical devices, cookware, food, grooming aids and agricultural biocides, and the like. Some of these compositions and formulations are well suited for use in wound dressings. Some of the formulation can kill and/or inhibit the growth of pathogenic bacteria, fungi, spores and viruses. The formulations comprise a compound such as an osmoticum in high enough concentration to create an osmotic gradient and at least one compound that acts to comprise the integrity of a microorganism's membrane or cell wall. These formulations may optimally include at least one agent that thickens the formulations. In some aspects the formulation is in the form of an emulsion.

### PRIORITY CLAIM

The application claims the benefit to U.S. provisional patent application No. 61/391/710 filed on Oct. 11, 2010, and incorporated herein by reference to its entirety.

### FIELD OF THE INVENTION

Aspects of the present invention relate to general antimicrobial compositions with emphasis on wound treatments.

### BACKGROUND

Wound healing is the process of tissue regeneration that restores function to skin defects. However, wound infection stalls the repair process by prolonging the inflammatory stage, preventing progress towards proliferation and remodeling. The end result is longer healing times, increased patient discomfort, reduced quality of life, disfiguring scar formation and potentially increased mortality. Current approaches to treat wound-level infections rely on antibiotics or potentially cytotoxic antimicrobials, like silver or iodine. The world-wide increase in antibiotic-resistant strains of bacteria like methicillin-resistant Staphylococcus aureus (MRSA), however, is beginning to diminish the applicability of antibiotics. Alternatively, antimicrobials such as silver and iodine have been shown to impair the orchestrated process of wound healing, leading to greater scar formation and delayed wound closure. Finally, bacterial resistance to commonly employed antimicrobial agents like silver ions has been well documented and clinically cited.

Accordingly, there is a need for an economical and effective wound dressing that helps to prevent or treat infected wounds; some aspects of the present invention address this need.

### SUMMARY

The present invention relates to antimicrobial compositions comprising a membrane permeabilizing entity that works synergistically with a hyperosmotic component and/or method of using the same. Preferred embodiments are well suited for use as medical dressings, antiseptics, cosmetic formulations and even as food additives. Some compositions are further capable of treating wound-level infections, promoting the wound healing process and/or reducing the morbidity, inflammation and scarring associated with wound management.

Preferred embodiments of the invention make use of a mechanism of antimicrobial action termed “osmopermeation”. Briefly, osmopermeation represents a cell-level biomechanical phenomenon that may lead to eradication of microscopic pathogens (bacteria, fungi, spores, viruses and the like). Osmopermeation may include a two-step coupled process of (i) disruption of pathogenic cell surfaces/membranes and (ii) subsequent dehydration of the organism via hyperosmotic stress. The two processes may work in tandem yielding a synergistic reaction, as the combined potency of both membrane disruption and osmotic imbalance are much greater than one would expect from simply summing their unitary effects.

Some aspects of the invention provide antimicrobial formulations and compositions, comprising: a membrane permeabilizing entity; and a hypertonic component, wherein the hypertonic component includes at least one saccharide or polyol. Some embodiments also include at least one thickening agent such as alginate while others may be formulated as oil in water emulsions. In some embodiments the hyperosmotic component includes at least one non-reducing saccharide selected from the group consisting of: sucrose (Saccharose; β-D-fructofuranosyl-(2→1)-α-D-glucopyranoside; β-(2S,3S,4S,5R)-fructofuranosyl-α-(1R,2R,3S,4S,5R)-glucopyranoside), glucose, fructose, lactose, mannose and dextrose. In still other embodiments the hyperosmotic component may be at least one polyol selected from the group consisting of: sorbitol, glycerol, glycol, arabitol, lactitol, ribitol, dulcitol, mannitol, maltitol, xylitol and isomalt. In still other embodiments the hyperosmotic component may be at least one carbohydrate, ester, salt or ionic solute. In these formulations the membrane permeabilizing agents the hypertonic reagents are present in the formulations in sufficient quantities to act in together with one another as bacteriastats or bactericides. The levels of the individual compounds in the inventive formulations are generally lower than they would be if each were used in the absence of the other to destroy or control the growth of most common microbes including various pathogenic bacteria.

In some embodiments of the invention the membrane permeabilizing entity in the composition or formulations is selected from the group consisting of: cationic agents, surface-active agents, chelators, iron-binding proteins and biguanides. In some embodiments the cationic agent is selected from the group consisting of: ions, polyelectrolytes and polycations. In still other embodiments the cationic agent is selected from the group consisting of chitosan, water soluble chitosan, chitosan derivatives, poly-l-lysine, polyethylenimine and diethylaminoethyl-dextran. In some embodiments the surface-active agent in the formulation is a surfactant, selected from the group consisting of: cations, anions, amphoteric moieties, and non-ionic or zwitterionic compounds. And in still other embodiments the surface-active agent is a quaternary ammonium compound selected from the group consisting of: benzalkonium chloride, benzethonium chloride, methylbenzethonium chloride, cetalkonium chloride, cetylpyridinium chloride, cetrimonium, cetrimide, dofaniumchloride, etraethylammonium bromide, and domiphen bromide. In yet other embodiments the surface-active agent is at least one compound selected from the group consisting of: phenols, phenolic derivatives, cresols, terpenes, terpenoids, phenylpropanoids and polychloropheoxy phenols. The surface-active agents may be is selected from the group consisting of: eugenol, thymol (2-Isopropyl-5-methylphenol), eucalyptol, malaleuca, carvacrol or cinnamaldehyde. In some embodiment the surface-active agent in the formulation may be selected from the group consisting of: plant derived volatile oils and derivatives of plant derived volatile oils. In some embodiments that include a chelator, the chelator may include at least one compound selected from the group consisting of: ethylenediaminetetra acetic acid, citric acid, gluconic acid, malonic acid or polyphosphates. In some embodiments that include an iron binding protein the protein may be selected from the group consisting of: lactoferrin, transferrin, and lactoferricin B. In some embodiments that include a biguanide it is selected from the group consisting of chlorhexidine, alexidine, vantocil, and polyhexamethylene biguanides.

In some formulations the membrane permeabilizing entity is included in an emulsion. In still other embodiments at least one membrane permeabilizing entity in the formulation is included in a suspension as a particle in oil in water (o/w) emulsion, nanoparticle, micelle or similar form. In some embodiments the particle size is on the micron scale within a range of about 1 μm to about 50 μm. And in still other embodiments the formulation includes particles sized is on the submicron scale within a range of between about 50 nm to about 1000 nm. Some embodiments are formulations in which the membrane permeabilizing entity is further activated to interact with a bacteria cell membrane by contact with bodily fluids. In some embodiments the inventive formulations include at least one oil. The oil may be a charged or uncharged fatty acid with carbon chain length between 14-22 carbon atoms and includes 0-6 double bonds. In some embodiments the surface-active agent helps to form an emulsion. In some embodiments the emulsions may include at least one positively charged moiety. In some embodiments the positively charged moiety is a quaternary ammonium compound.

In some embodiments the inventive formulations further include at least one additional antimicrobial agent; in some embodiments these agents may be associated with an emulsion, additional antimicrobial agents may be selected from the group consisting of: silver, selenium, and antibiotics. In some of the inventive formulations the additional antimicrobial compound acts synergistically with the hyperosmotic and/or membrane permeabilizing entity.

In some embodiment the inventive formulation includes at least one hyperosmotic component is selected from the groups consisting of: a saccharide component with an osmolarity between about 0.45 OsM to about 4.5 OsM; or a polyol component having an osmolarity between about 0.25 OsM to about 10.0 OsM; or a saccharide and polyol mixture having an osmolarity of between about 0.25 OsM to about 10.0 OsM. In some embodiments the antimicrobial formulation has a water activity of between about 0.42 to about 0.99. In some embodiments the formulation is well suited for use a wound dressing. In some embodiments the inventive formulation is in an aqueous state, and the concentration of the membrane permeabilizing entity in the formulation is between about 0.0001% to about 4.5% (w/w) of the entire formulation. In some embodiments the inventive formulation is in a semi-solid state, and wherein the concentration of the hyperosmotic component is between about 40% to about 99.999% (w/w) of the entire formulation. In still other embodiments the inventive formulation is in a colloidal state, wherein the concentration of the membrane permeabilizing entity is between about 0.00001% to about 5% (w/w) of the entire formulation. In yet other embodiments the inventive formulation is in a solid or colloidal state, and wherein the concentration of the hyperosmotic component in the formulation is between about 40% to about 99.999% (w/w) of the entire formulation. In some embodiments the concentration of the membrane permeabilizing entity in the inventive formulation entity is present in the formulation in a range between about 0.00001% to about 5% (w/w) of the entire formulation.

In some embodiments the inventive formulations include at least one absorbent agent. In some embodiments the absorbent agent in the formulation is, or is derived from, at least one compound selected from the groups consisting of: alginic acid or salt thereof; carrageenan; a derivative of alginic acid or salt thereof and a derivative of carrageenan. In some embodiments the inventive formulation is in the form of a liquid, pliable solid, semi-solid paste, gel, expanding foam, hydrocolloid, ointment, or cream. In still other embodiments, the inventive formulations are provided in combination with a non-woven fibrous pad. In some embodiments the pad in contact with the formulation is created via a melt-spun process and extrusion through a fibrous mesh to produce pliable fibers. In some embodiments the inventive formulation is in contact with a pad wherein the pad has at least one adhesive border, in some embodiments the pad may have no adhesive borders.

In still other embodiments the inventive formulations include at least one compound such as a dye that changes color when the wound conditions change. Changes can be related to wound moisture, pH or temperature.

Yet other embodiments of the invention include inventive antimicrobial formulations that can be used to reduce the number of microorganisms. Still other embodiments include methods of use in the inventive formulation to reduce the number of microbes on a surface. These methods may comprise the steps of: contacting a surface with any one of the inventive formulations disclosed herein or reasonably inferred from the claims and description provided herein. In embodiments the inventive formulation is contacted with a site selected from the group of sites consisting of: acute traumatic wounds; chronic non-healing wounds; recovering skin grafts; pressure ulcers; surgical insults; skin deformities; cancerous lesions; oral wounds; acne; viral infections; and fungal infections.

### BRIEF DESCRIPTION OF THE FIGURES

FIG. 1A. A cartoon illustrates the interaction between an emulsion that incorporates at least one membrane permeabilizing agent and bacterial cells.

FIG. 1B. A cartoon illustrating a plausible mechanism for how osmopermeation may act to destroy bacterial cells.

FIG. 2A. A scanning electron-micrograph of untreated bacteria cells.

FIG. 2B. A scanning electron-micrograph of bacteria cells treated with a membrane permeabilizing agent (6.6 mM thymol) and a hyperosmotic gradient (1.17M Sucrose).

FIG. 2C. A scanning electron-micrograph of bacteria cells exposed to a formulation that causes the cells to develop blebs. These blebs are indicative of membrane and cell damage.

FIG. 2D. A scanning electron-micrograph of bacteria cells exposed to a formulation that causes the cells to develop blebs and other membrane artifacts. These structures are indicative of cell membrane damage.

FIG. 2E. A bar graph shown the % E. coli that are intact or that exhibit Blebs or evidence of cell disruption measured for untreated cells and for cells treated with an inventive formulation.

FIG. 2F. A graph of L/D ratios measured with both treated and untreated cells.

FIG. 3A. Photomicrographs of cells that were: untreated; or treated with sucrose, or thymol or sucrose and thymol (S+T).

FIG. 3B. Graphs of frequency versus pixel intensity measured for cells that were: untreated (tope right); treated with sucrose (S), top left; Thymol (T) bottom right; or Sucrose and Thymol (S+T), bottom right.

FIG. 3C. Bar graphs showing % fluorescence intensity of cells that were: untreated, or treated with: sucrose (S); thymol (T); or sucrose and thymol (S+T); or QAC.

FIG. 3D. Graph of arbitrary absorbance units measures over wavelengths from 200 nm to 400 nm for cells were: untreated, or treated with: sucrose (S); thymol (T); or sucrose and thymol (S+T); or QAC.

FIG. 4.A. Bar graph of intracellular normalized ATP units measured for cells that were untreated, or treated with: sucrose (S); thymol (T); or sucrose and thymol (S+T); or QAC for either 10 or 60 minutes.

FIG. 4.B. Photomicrographs of cells at times 0 through 60 minutes after treatment with sucrose (S); thymol (T); or sucrose and thymol (S+T); or QAC

FIG. 5 A. Graphs of Fa values as a function of dose measured with E. coli cells (left panel) or E. faecalis cells (right panel) treated with different doses of Sucrose (M), Thymol (mM) or Sucrose plus Thymol (S+T).

FIG. 5 B. Graphs of Fa values as a function of dose measured with S. aureus cells (left panel) or MRSA cells (right panel) treated with different doses of Sucrose (M), Thymol (mM) or Sucrose plus Thymol (S+T).

FIG. 5 C. Plots showing antagonism between Sucrose (S) and Thymol (T) measured with treated E. coli cells (left panel) and E. faecalis cells (right panel).

FIG. 5D. Plots showing antagonism between Sucrose (S) and Thymol (T) measured with treated S. aureus (left panel) and MRSA cells (right panel).

FIG. 6A. Graph of wound area % over days measured with wounds that were treated with the inventive formulation of osmopermeation (OPT), 1% silver-sulfadiazine cream (SSD) or not treated (control).

FIG. 6B. Photographs of wounds from day 0 to day 10 after wounding; left column shows wounds not treated with the inventive formulation (Control), middle column shows wounds treated with 1% silver-sulfadiazine cream (SSD) and the right column shows wounds treated with the inventive formulation of osmopermeation (OPT).

FIG. 6C. 3-D reconstructions of 8 mm healed lesion sites for control and osmopermeation (OPT) treated animals after 14 days.

FIG. 6D. Representative transdermal scans of wounds after 14 days of treatment with either standard treatment (control), 1% silver-sulfadiazine cream (SSD), or the inventive formulation of osmopermeation (OPT).

### DESCRIPTION

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one of ordinary skill in the art to which this invention pertains.

As explicitly stated or clearly implied otherwise as used herein the term ‘about’ refers to a range of values of plus or minus 10 percent, for example, about 1.0 includes the range of values 0.9 to 1.1.

Recently, there has been re-evaluation in the use of hyperosmotic gradients (generated by saccharides or honeys) as alternatives to caustic antimicrobials. Hyperosmotic compounds are thought to act on two biologic levels (i) on the cellular scale to hinder microbial reproduction and (ii) at the tissue level whereby the osmotic gradients actively enhance microcirculation, moderate wound pH and improve debridement.

However, hyperosmotic therapies have several drawbacks that include lack of potency (bacteriostatic), slow kinetics of action (requires hours to days for infection control) and impractical forms of delivery. For instance, while bacterial DNA synthesis and replication are stalled in hyperosmotic environments, many strains of bacteria remain highly viable even at osmotic pressures near 10 MPa, which is estimated to be the structural limit of the cell wall. Other adaptive cell processes include the synthesis or importation of compatible solutes, which effectively reduce the net transmembrane osmotic potential. These survival mechanisms demonstrate that hyperosmotic saccharides are bacteriostatic and not bactericidal.

Furthermore, deficient means of delivery prohibit widespread application of hyperosmotic treatments in clinical and non-clinical market applications. For instance, medical records indicate the effectiveness of simple granulated sucrose or raw honey in wounds, but emphasize the impractical nature of applying the substance. Often the use of granulated sugar or raw honey requires the patient to remain in a supine position to circumnavigate application impediments caused by gravity.

Despite acknowledgement of some clinical benefits offered by hyperosmotic wound environments, practical biological issues limit widespread treatment adoption in both clinical and non-clinical settings.

To rectify the issues of potency, delivery and slow kinetics of hyperosmotic microbial action, the inventors have developed a bactericidal formulation that significantly enhances the positive aspects of hyperosmotic gradients. The method through which these formulations act can be referred to as “osmopermeation”. The innovative osmopermeation compositions or formulations utilize membrane permeabilizing entities in tandem with hyperosmotic gradients to rapidly and critically dehydrate pathogenic microbes. The present invention describes the means to target the bacterial membrane and concentrations that synergistically amplify the deleterious biologic effects of hyperosmotic stress.

As exemplified herein, work by the inventors demonstrates that intentional membrane disruption in combination with a hyperosmotic gradient exhibits synergistic wide spectrum bactericidal potency. Synergy is defined by Chou et al. Pharmacological Rev. 58: 621-681, 2006, “Theoretical Basis, Experimental Design, and Comouterized Simulations of Synergism and Antagonism in Drug Combination Studies. Indeed, when bacteria are exposed to hyperosmotic gradients in the presence of a membrane permeabilizing agent, the microbial dehydration process is amplified by several orders of magnitude. Moreover, the biocidal effect often occurs on the scale of seconds to minutes. This synergistic nature of the combinations are especially effective when the concentrations of the constituents in the formulations are present at levels that would be considered sub-lethal if the compounds were used separately from one another. For example, a 1.17 OsM of hypertonic sucrose has no bactericidal effect and actually permits cell proliferation. Similarly, a sub-lethal concentration of a membrane permeabilizing agent such as thymol has no significant bactericidal effect. However, the same growth tolerant sucrose solution (1.17 OsM) in conjunction with the sub-lethal concentration of a membrane permeabilizing agent induces 8 logs of bactericidal action. This bactericidal activity is a 10-100 fold increase as to what is predicted by individually summing the component effects. Thus, the bacteriostatic nature of hyperosmotic stress is converted into a bactericidal state in the presence of a membrane permeabilizing compound. This is a dramatic and unexpected finding. Without being bound to any single theory or limiting explanation of the invention this synergy may be a function of osmotic pressure and membrane fluidity, which in turn is directly correlated to component dosages (i.e. synergy only occurs within a range of concentrations). For example, sugars in compositions with osmotic pressures on the order of <0.45 OsM may be quickly metabolized and fuel growth. In contrast, extremely high concentrations of sugars with osmotic pressures on the order of >4.5 OsM may increase the viscosity of the formulation to the extent that it adversely effects binding affinity of membrane permeabilizing agents to the microbial cell membrane. Accordingly, preferred embodiments of the invention include optimum synergistic combinations of membrane permeabilizing agents and hyperosmotic agents delivered in a form that facilitates pathogen membrane fusion and subsequent cellular dehydration. Bacteria strains the invention is effective against include, but is not limited to: Escherichia coli, Staphylococcus epidermidis, methicillin-resistant Staphylococcus epidermidis Enterococcus faecalis, Staphylococcus aureus, Pseudomonas aeruginosa, Streptococcus mutans, Salmonella choleraesuis, Enterobacter aerogenes. Fungus the invention is effective against include but is not limited to: Neurospora crassa, Aspergillus nidulans, Candida albicans, Magnaporthe grisea, Pichia pastoris, Saccaromyces cerevisiae and Schizosaccharomyces pombe.

Additionally, these optimal sub-lethal dosage combinations have been shown to possess limited toxicity to host tissue and therefore, osmopermeation does not interfere with the complex wound healing process. Indeed, the inventors have shown in a preclinical animal model that osmopermeation effectively eradicates wound-level bacteria and simultaneously accelerates wound healing in comparison to standard treatments like ionic silver.

Examples of membrane permeabilizing agents that can be used to practice the invention include phenolic compounds, such as 2-isopropyl-5-methylphenol (thymol). Thymol has known membrane disrupting properties and exhibits free-radical scavenging capabilities, making it an attractive agent in wound management. The amphiphilic nature of thymol allows for the formation of emulsions that may increase fusion with bacterial membranes to further disrupt cell membrane integrity. Thymol-based emulsions can be manufactured through oil-in-water (o/w) homogenization techniques. Further, preferred embodiments tune the emulsion with surfactants, alcohols, lipids and/or fatty acids for specific applications. For instance, surfactants or charged lipids can be used to stabilize the emulsion and control interaction with cellular membranes by imparting a net charge on the surface of the emulsion particle. The net surface charge increases emulsion stability by preventing coalescence through electrostatic repulsion. Addition of an anionic or cationic surfactant creates a repulsive system that prevents nanosphere interaction to inhibit agglutination. Conversely, addition of surfactants to create net surface charge could be used to ensure preferential binding of emulsion particles with microbial membranes. The surfaces of microbes are typically negatively charged; therefore emulsion particles that incorporate positively charged cationic surfactants or cationic lipids would preferentially fuse through an electrostatic attraction. Negatively charged nanospheres that incorporate anionic surfactants would conversely be expected to repel negatively charged cellular membranes. Consequently, controlling the surface charge characteristics of the emulsion delivery system effectively increases stability, may minimize non-specific fusion and/or improve preferential fusion with microbial surfaces. Other embodiments regulate particle size by increasing the amount of free energy deposited in the system during the manufacturing process to further decrease emulsion particle size to range between the nano-scale (nanometers—nm) and micro-scale (micrometers—μm).

Still other embodiments include alternative membrane permeabilizing agents, such as cationic polypeptides, surfactants, chelators, electrolytes, ions, quaternary ammonium compounds, volatile oils or antibiotics capable of targeting the bacterial membrane. Upon fusion, the membrane permeabilizing agent disrupts orderly packing of the cell membrane to prevent osmoregulation.

In one embodiment, a metal chelator such as EDTA or citric acid is used to enhance the emulsion effectiveness on bacterial membranes. The chelator works to bind aqueous metal ions and thus free bacterial surface binding sites that may be occupied by the metal ions.

In still other embodiments, naturally or petroleum derived lipids can be used as a carrier for the emulsion. These lipids can be saturated or unsaturated and have a carbon chain length of 14-22 bonds.

Additional agents with synergistic drug effects can be, glucose oxidase that catalyzes oxidation of glucose to form hydrogen peroxide, which targets the structural integrity of the bacterial cell membrane and/or methylglycoxal a naturally produced aldehyde that exhibits non-peroxide based antibiotic activity and/or nanocrystalline or ionic silver. Still other compounds can be included in the preparations such as additional drugs, antimicrobials, analgesics, and/or compounds that affect the viability of bacteria and/or promote wound healing and patient comfort.

Once the emulsion has been formed, osmotic agents including diverse compounds such as basic carbohydrates, saccharides, esters, polyols, salts and ionic solutes may be added in concentration to create an osmotic pressure and to form a composition that has a paste or gel-like consistency. In use, the paste is contacted with a wound either by direct application of the paste or gel onto the wound or by applying the paste or gel to a gauze or other support such as a bandage.

Some embodiments include a trio of agents (i.e., emulsion, saccharides and alginate), hereinafter referred to as the stock compound, that can be used as a delivery vehicle for additional agents that would increase the efficacy of the generic composition and further promote healing through synergistic effects (i.e., interaction in ways that enhance or magnify one or more effects of both the stock compound described above and the additional agents).

Some embodiments include alginate a highly absorbent polysaccharide that readily binds water to form a viscous gel. Some embodiments include a level of sodium or calcium alginate or derivative of alginic acid sufficient to form a hydrocolloid alginate dressing. The inclusion of alginate in wound dressings of the present invention enables the dressing to absorb large amounts of exudates, effectively cleaning the wound and trapping excess water to maintain a moist environment, properties that aid in rapid healing. A moist wound environment supports the wound healing process by encouraging autolytic debridement (i.e., breakdown of all or part of a cell or tissue by self-produced enzymes), enabling granulation to proceed under optimum conditions. In part because these dressings can absorb more moisture than more conventional dressings, dressings that use these formulations may not need to be changed as often as more conventional dressings.

One of the primary applications for osmopermeation is topical medical wound dressings. Still other applications for the inventive composition, formulations and methods of invention include their uses as general antimicrobial agents (i.e. disinfectants, food additive or coating, medical coatings, liquid flushes, etc.), anti-viral agents, anti-fungal agents, intranasal delivery vehicle or chemotherapeutic agents suitable for ingestion or internal administration for the treatment of disease.

Further advantages of the present invention will become evident from the following description and examples. Exemplary compositions given along with the corresponding percentage of the total mass of the dressing (weight/weight %). Aqueous and semi-solid compositional forms are as shown.

TABLE 1
Highly Viscous Formulation for Treating Wounds

| Component | Weight/Weight (%) |
|---|---|
| Sugar | 71.0 |
| Polycation | 1.0 |
| Alginate | 2.5 |
| Water | 25.5 |
| Total | 100 |

Briefly the formulation of Table 1 can be created by adjusting the pH of an aqueous solution to 6 using for example an organic acid. Next, a polycation (e.g. Chitosan) and the thickener alginate are dissolved in the water and mixed until a gel begins to form. Finally, extremely fine grained (e.g. powdered sugar (Sucrose) is added to the gel.

The osmopermeation formulations can be supplement by the addition of a variety of components that have antimicrobial and/or therapeutic properties. Compounds that can be added to the formulations include, but are not limited to hydrophobic phenolic moiety.

Still other compounds that can be added to the formulations include, but are not limited to the enzyme glucose oxidase which catalyzes the production of hydrogen peroxide or compounds such as methylglycoxal (MGO), a naturally occurring aldehyde that possess non-peroxide based antibiotic activity. Still other additives include metal such as elemental silver and any number of a variety of antibiotics, including for example, bacitracin, neomycin, polymixin b and the like.

TABLE 2
Example 1

| Component | Mass (g) | Weight/Weight (%) |
|---|---|---|
| Fructose | 45 | 45 |
| Sucrose | 45 | 45 |
| Thymol | 0.01 | 0.01 |
| Water | 9.99 | 9.99 |
| Total | 100 | 100 |

TABLE 3
Example 2

| Component | Mass (g) | Weight/Weight (%) |
|---|---|---|
| Fructose | 45 | 34.6 |
| Sucrose | 45 | 34.6 |
| Glucose | 30 | 23.08 |
| Thymol | 0.01 | 0.0077 |
| Water | 9.99 | 7.68 |
| Total | 130 | 100 |

TABLE 4
Example 3

| Component | Mass (g) | Weight/Weight (%) |
|---|---|---|
| Fructose | 45 | 45 |
| Sucrose | 45 | 45 |
| Chitosan | 0.0008 | 0.0008 |
| Water | 9.992 | 9.992 |
| Total | 100 | 100 |

TABLE 5
Example 4

| Component | Mass (g) | Weight/Weight (%) |
|---|---|---|
| Fructose | 40 | 36.36 |
| Sucrose | 40 | 36.36 |
| Sorbitol | 25 | 18.18 |
| Thymol | 0.01 | 0.0091 |
| Water | 9.99 | 9.081 |
| Total | 110 | 100 |

TABLE 6
Example 5

| Component | Mass (g) | Weight/Weight (%) |
|---|---|---|
| Fructose | 40 | 36.36 |
| Sucrose | 40 | 36.36 |
| Glucose | 20 | 18.18 |
| Triton-X | 0.1 | 0.09 |
| Water | 9.9 | 9.00 |
| Total | 110 | 100 |

TABLE 7
Example 6

| Component | Mass (g) | Weight/Weight (%) |
|---|---|---|
| Fructose | 45 | 45 |
| Sucrose | 45 | 45 |
| Benzethonium Chloride | 0.0005 | 0.0005 |
| Calcium | 0.01 | 0.01 |
| Water | 9.9895 | 9.9895 |
| Total | 100 | 100 |

TABLE 8
Example 7

| Component | Mass (g) | Weight/Weight (%) |
|---|---|---|
| Fructose | 45 | 45 |
| Sucrose | 45 | 45 |
| Benzethonium Chloride | 0.0005 | 0.0005 |
| Eicosapentaenoic Acid | 2 | 2 |
| Ascorbic Acid | 0.01 | 0.01 |
| Thymol | 0.01 | 0.01 |
| Water | 7.9795 | 7.98 |
| Total | 100 | 100 |

TABLE 9
Example 8

| Component | Mass (g) | Weight/Weight (%) |
|---|---|---|
| Fructose | 40 | 36.36 |
| Sucrose | 40 | 36.36 |
| Glucose | 20 | 18.18 |
| Thymol | 0.01 | 0.0091 |
| Alginate | 1 | 0.91 |
| Water | 8.99 | 8.17 |
| Total | 110 | 100 |

TABLE 10
Example 9

| Component | Mass (g) | Weight/Weight (%) |
|---|---|---|
| Fructose | 45 | 40.91 |
| Sucrose | 45 | 40.91 |
| Glycol | 10 | 9.09 |
| Benzethonium Chloride | 0.001 | 0.00091 |
| Formic Acid | 0.01 | 0.091 |
| PHMB | 0.02 | 0.01818 |
| Water | 9.969 | 9.06 |
| Total | 110 | 100 |

TABLE 11
Example 10

| Component | Mass (g) | Weight/Weight (%) |
|---|---|---|
| Sucrose | 10 | 49.98 |
| Benzethonium Chloride | 0.001 | 0.005 |
| Sodium dodecyl sulfate | 0.01 | 0.05 |
| PHMB | 0.02 | 0.10 |
| Water | 9.979 | 49.87 |
| Total | 20.01 | 100 |

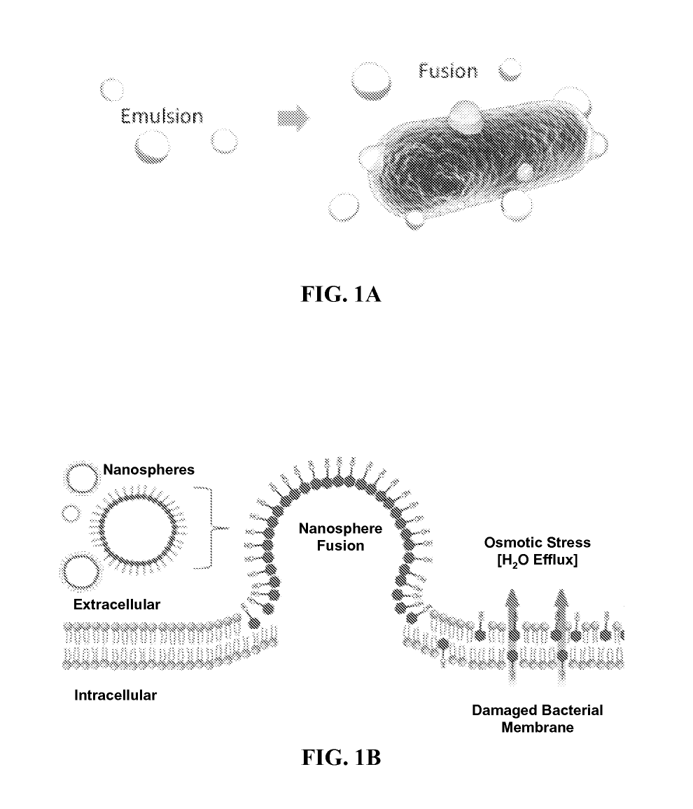

Referring now to FIGS. 1, 2, 3, 4, 5, and 6 the mechanism of bactericidal activity is depicted. Specifically, FIG. 1A depicts the fusion process for an emulsion consisting of at least one membrane permeabilizing agent. The pictorial shows the emulsions attach to the bacterial surfaces via electrostatic or hydrophobic interactions. FIG. 1B denotes the mechanism of action when bacteria are subjected to both the membrane permeabilizing agent and hyperosmotic stress. As the membrane permeabilizing agent increases the fluidity of the lipid layers, the bacteria's ability to regulate the imposed osmotic potential decreases. As a result, water and intracellular contents leak into the extracellular space. This dehydration process is accompanied by cell shrinkage, plasmolysis and eventual death.

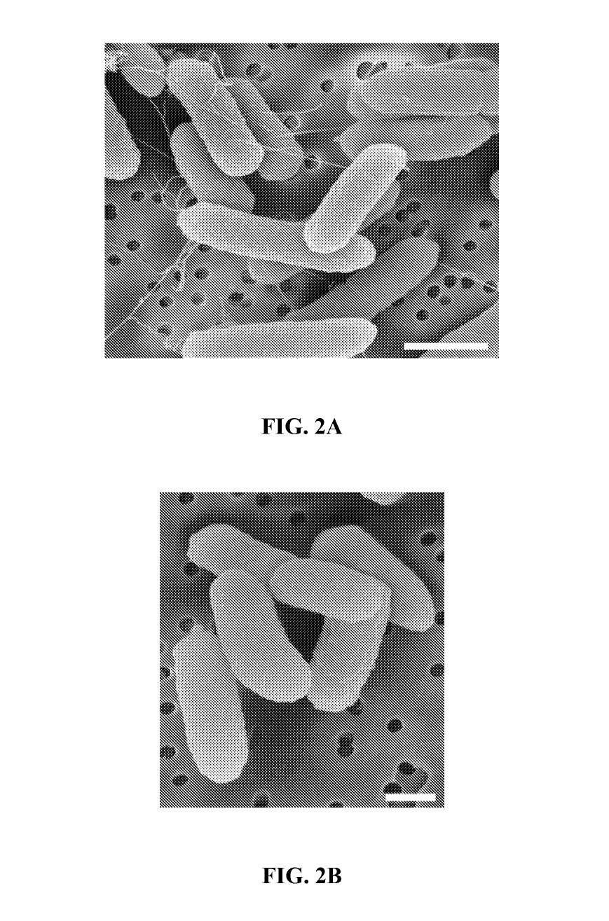

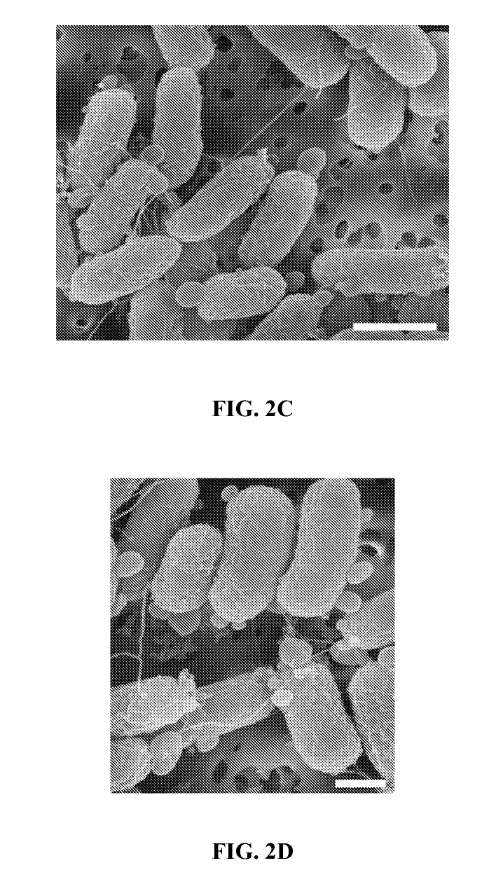

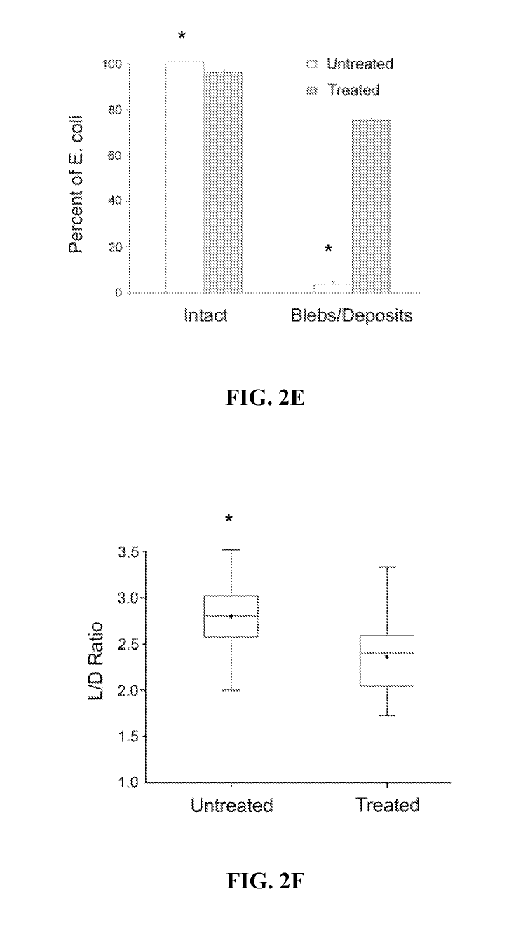

Referring now to FIG. 2, attributes of various compositions disclosed herein can be readily observed in these scanning electron micrographs. The photomicrographs of treated Escherichia coli show signs of decreased cell volume, roughened surface texture, membrane blebbing and lysis.

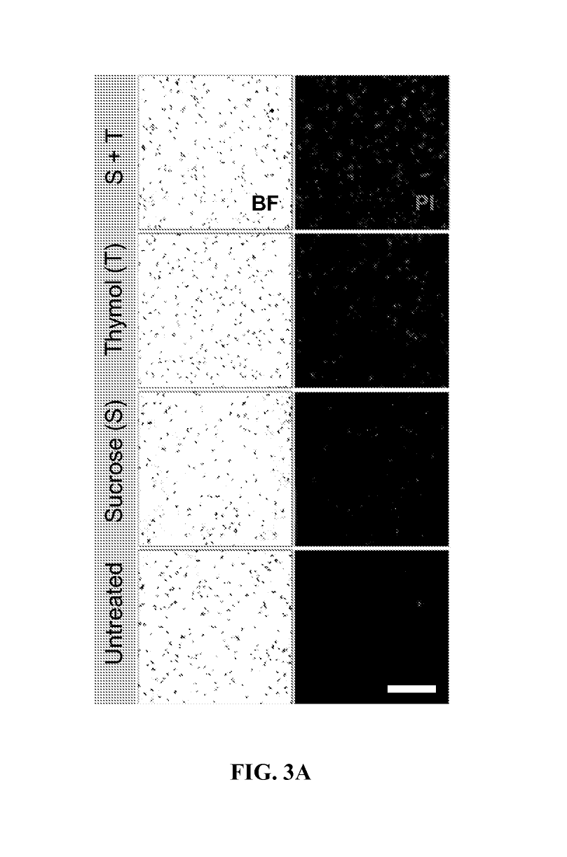

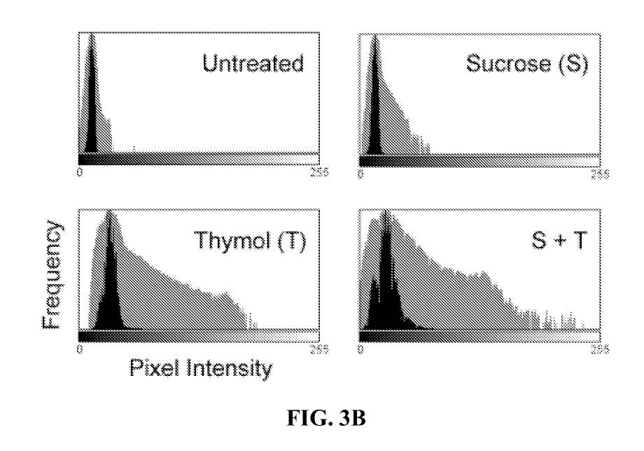

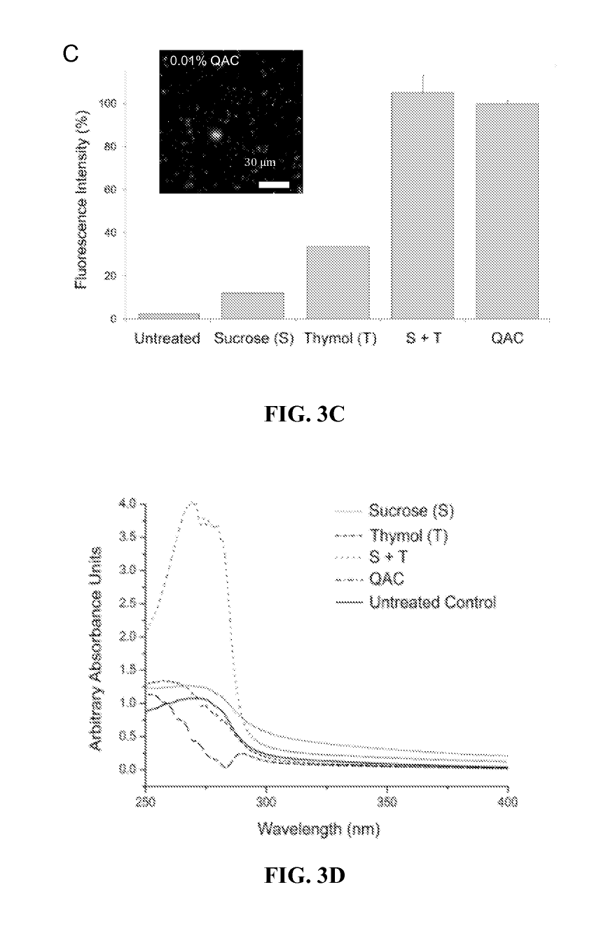

Referring now to FIG. 3, this figure further demonstrates how a membrane permeabilizing agent (i.e. thymol) enhances the bactericidal action of sucrose. Applied singularly, both thymol (2.66 mM) and sucrose (1.17 OsM) have little effect on cell viability when using a vital stain (propidium iodide, PI). However, when both agents are combined, there is significant amplification of cell death after 1 hr. Subsequent measurement of 260/280 nm absorbing material show that the combination also enhances bacteriolysis and loss of intracellular proteins (4-fold increase over each component). Table 1 summarizes some of the data shown in FIGS. 3A to 3D.

TABLE 12

| | 260 nm | 280 nm | 260/ |
|---|---|---|---|
| | 9(A.U.) | (A.U.) | 280 nm |
| Untreated | 1.01 | 1.0 | 1.01 |
| Sucrose (S) | 1.19 | 1.03 | 1.15 |
| Thymol (T) | .99 | 1.02 | 0.97 |
| S + T | 3.5 | 4.1 | 0.85 |
| QAC | — | — | — |

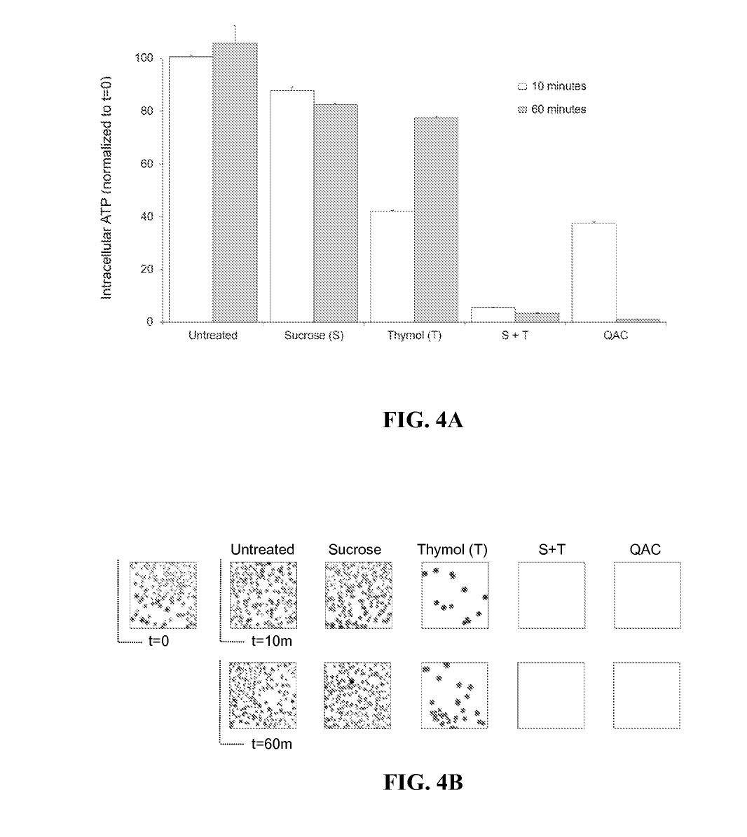

Referring now to FIG. 4, the bactericidal effect of osmopermeation is fast acting and occurs in less than 10 minutes, as shown. Finally, osmopermeation is wide spectrum in nature and effective in treating both Gram (+) and Gram (−) bacterial strains. Table 2 includes a summary of the data presented in FIGS. 4A and 4B.

TABLE 13

| | Untreated | Sucrose (S) | Thymol (T) | S + T | QAC |
|---|---|---|---|---|---|
| Log Red - 10 min | Growth | <0.5 | ~6.4 | >8.0 | >8.0 |
| Log Red - 60 min | Growth | <0.2 | ~6.7 | >8.0 | >8.0 |

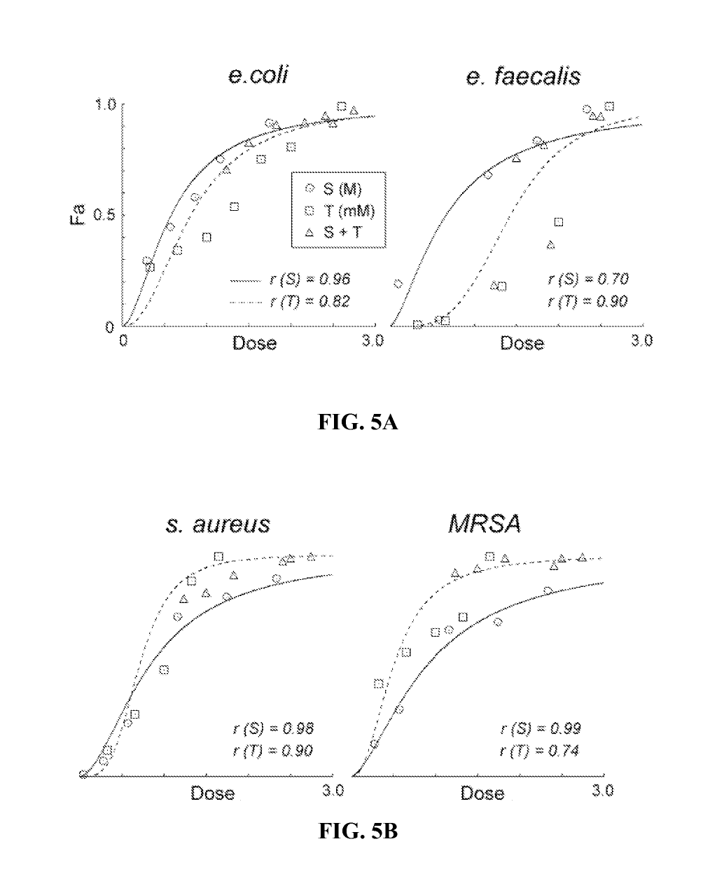

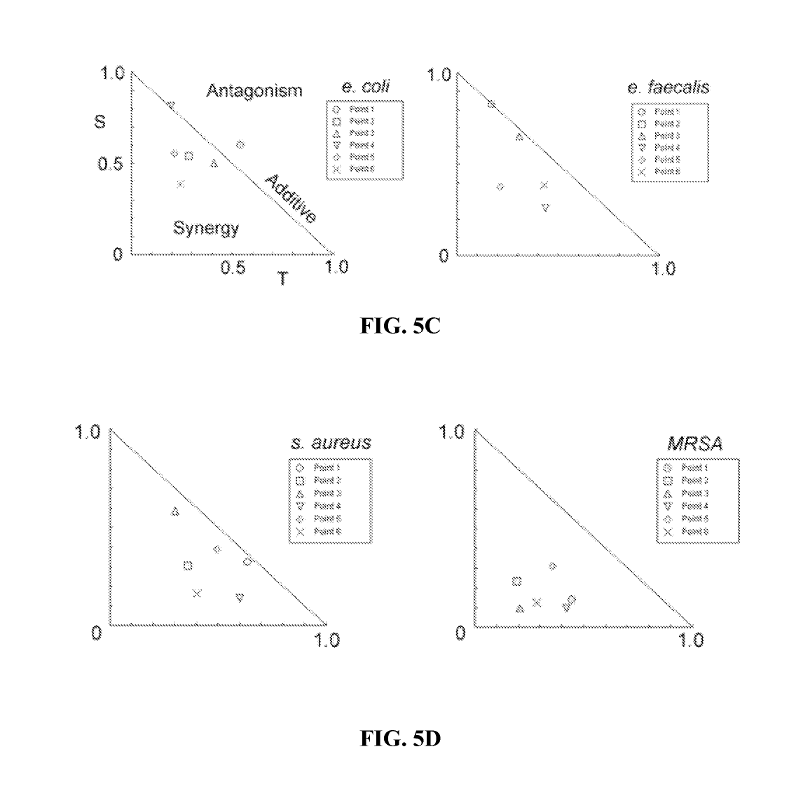

Referring now to FIG. 5, dose response curves of four strains of bacteria including MRSA to hyperosmotic sucrose (S, units: M) and thymol emulsion (T, units: mM) are provided. Responses represent 24 hrs of treatment. Six specific combinations (S+T) of sucrose and thymol are also plotted for each strain. The effect level (Fa) represents a value between 0 and 1.0 with 0 being no effect and 1.0 being all cells affected. (B) Normalized isobolograms defining the interaction between S and T. Numbered points correspond to the specific combinations enumerated in the table. Table denotes the minimum inhibitory concentrations (MICs) for each treatment as well as outcomes of the six S+T combinations. The nature of the two-treatment interaction is measured via the combination index (CI). The CI<1 is roughly defined as synergistic, ˜1 is additive and CI>1 is antagonistic. See Chou T C. “Theoretical basis, experimental design and computerized simulation of synergism and antagonism in drug combination studies”. Pharmacol. Rev. 2006; 68:621-81. Synergistic interactions were found in combinations with various hyperosmotic pressures and concentrations of the membrane permeabilizing agent.

Referring now to FIGS. 5A and 5B., graphs of Fa values measured as a function of dose of Sucrose (S), Thymol (T) or both Sucrose and Thymol (S+T). Data collected with 4 strains of bacterial E. coli, E, faecalis, S. aureus and MRSA. FIGS. 5C. and 5D., show antagonism plots (Sucrose (S) versus Thymol (T) measured for 4 different strains of bacteria E. coli, E, faecalis, S. aureus and MRSA. Tables 3, 4, 5 and 6 summarizing MIC_{S} and MIC_{T} values measured with different levels of Sucrose (S) or Thymol (T).

TABLE 14
values measured with E. coli.
e coli (MIC_{S} = 2.63M; MIC_{T} = 2.60 mM)

| Point | Dose S | Dose T | Fa | Cl |
|---|---|---|---|---|
| 1 | 0.58 | 0.66 | 0.71 | 1.14 |
| 2 | 1.17 | 0.66 | 0.91 | 0.83 |
| 3 | 1.17 | 1.00 | 0.92 | 0.91 |
| 4 | 1.17 | 0.33 | 0.83 | 1.02 |
| 5 | 1.75 | 0.66 | 0.95 | 0.77 |
| 6 | 1.75 | 1.00 | 0.97 | 0.64 |

TABLE 15
values measured with E. faecalis
e-faecalis (MIS_{S} = 2.63M; MIC_{T} = 2.60 mM)

| Point | Dose S | Dose T | Fa | Cl |
|---|---|---|---|---|
| 1 | 0.58 | 0.66 | 0.19 | 2.94 |
| 2 | 1.17 | 0.33 | 0.76 | 1.00 |
| 3 | 1.17 | 0.66 | 0.82 | 0.96 |
| 4 | 1.17 | 1.33 | 0.95 | 0.70 |
| 5 | 1.75 | 0.66 | 0.95 | 0.59 |
| 6 | 1.75 | 1.33 | 0.95 | 0.82 |

TABLE 16
values measured with S. aureus.
s-aureus (MIC_{S} = 2.92M; MIC_{T} = 1.65 mM)

| Point | Dose S | Dose T | Fa | Cl |
|---|---|---|---|---|
| 1 | 0.58 | 0.66 | 0.80 | 0.96 |
| 2 | 1.17 | 0.33 | 0.83 | 0.89 |
| 3 | 1.17 | 0.66 | 0.91 | 0.89 |
| 4 | 1.17 | 1.33 | 0.99 | 0.74 |
| 5 | 1.75 | 0.66 | 0.97 | 0.67 |
| 6 | 1.75 | 1.00 | 0.99 | 0.57 |

TABLE 17
values measured with MRSA.
MRSA (MIC_{S} = 2.92M; MIC_{T} = 1.65 mM)

| Point | Dose S | Dose T | Fa | Cl |
|---|---|---|---|---|
| 1 | 0.58 | 0.66 | 0.92 | 0.59 |
| 2 | 1.17 | 0.33 | 0.94 | 0.43 |
| 3 | 1.17 | 0.66 | 0.985 | 0.31 |
| 4 | 1.17 | 1.33 | 0.985 | 0.52 |
| 5 | 1.75 | 0.66 | 0.95 | 0.67 |
| 6 | 1.75 | 1.00 | 0.988 | 0.41 |

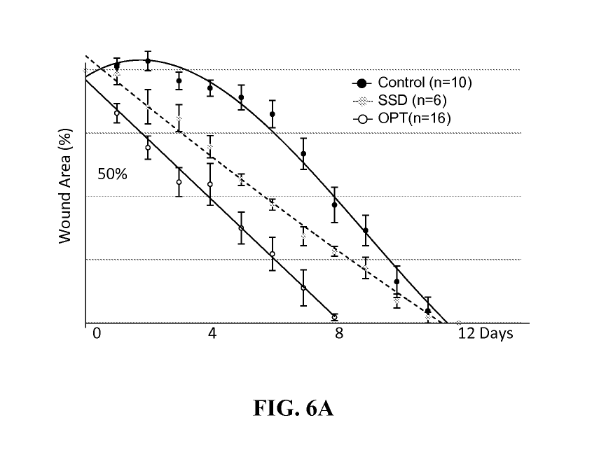

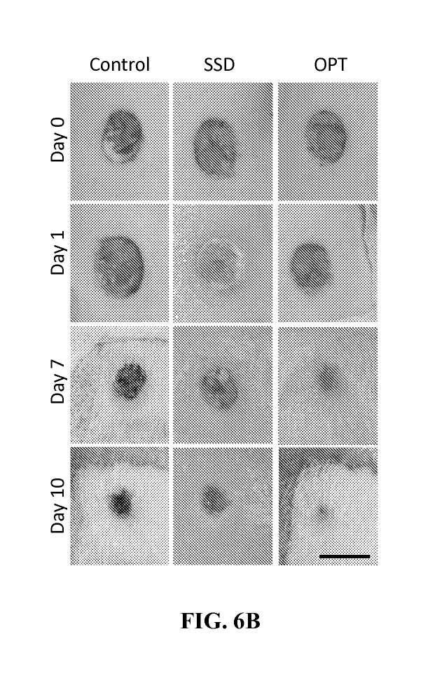

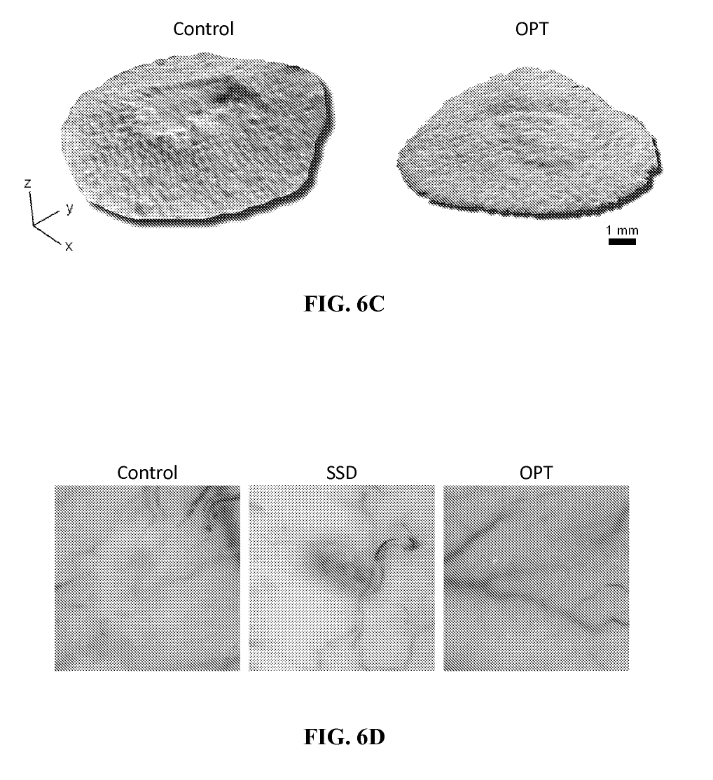

Referring now to FIG. 6A, a graph illustrating the improved wound healing capabilities of osmopermeation based dressings in an infected wound model. Using guinea pigs with an 8 mm full-thickness dermal injury, it was found that osmopermeation dressings possess low-toxicity, fights infection and increases the speed of healing. Reduced scarring is also evident in the infected wound model. Note: wounds were infected with Escherichia coli and Enterococcus faecalis strains at Day 0.

Referring now to FIG. 6B, Representative photographs of wounds measured in FIG. 6A, from day 0 to day 10 after wounding; left column shows wounds not treated with the inventive formulation (Control), middle column shows wounds treated with 1% silver-sulfadiazine cream (SSD) and the right column shows wounds treated with the inventive formulation of osmopermeation (OPT). Completed preclinical studies indicate that osmopermeation decreases healing time by approximately 3-4 days, facilitates capillary infiltration and minimizes scar formation in comparison to standard treatment using a polyurethane foam pad and occlusive dressing.

Referring now to FIG. 6C, representative 3-D reconstructions of 8 mm healed lesion sites for control and osmopermeation (OPT) treated animals. The 3-D geometry was created using close range photogrammetry. The data shows that after 14 days post-infection, the control side has significant cosmetic defects, peri-wound contraction lines and a large central depression. In contrast, treated animals show more complete tissue fill and fewer tension lines. This data is suggestive of improved wound healing and possible reduction in scar formation.

Referring now to FIG. 6D, representative transdermal scans of wounds after 14 days of treatment with either standard treatment (control), 1% silver-sulfadiazine cream (SSD), or the inventive formulation of osmopermeation (OPT). The data demonstrates enhanced tissue remodeling and increased blood vessel infiltration, indicative of decreased scar formation, in wounds treated with osmopermeation (OPT).

Those skilled in the art will recognize that numerous modifications can be made to the specific implementations described above. Therefore, the following claims are not to be limited to the specific embodiments illustrated and described above. The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others. All patents, patent applications, and references to texts, scientific treatises, publications, and the like referenced in this application are incorporated herein by reference in their entirety.

1. An antimicrobial formulation, comprising:
a membrane permeabilizing entity; and
a hypertonic component, wherein the hypertonic component includes at least one saccharide or polyol.

2. The formulation of claim 1, wherein the hyperosmotic component includes at least one compound selected from the group consisting of: sucrose, glucose, fructose, lactose, mannose, dextrose, polyols selected from the group consisting of: sorbitol, glycerol, glycol, arabitol, lactitol, ribitol, dulcitol, mannitol, maltitol, xylitol and isomalt.

3. (canceled)

4. The formulation of claim 1, wherein the membrane permeabilizing entity is selected from the group consisting of: cationic agents, surface-active agents, chelators, iron-binding proteins and biguanides.

5. The formulation of claim 4, wherein the membrane permeabilizing agent is selected from the group consisting of: ions, polyelectrolytes-, polycations, chitosan, water soluble chitosan, chitosan derivatives, poly-l-lysine, polyethylenimine and diethylaminoethyl-dextran.

6. (canceled)

7. The formulation of claim 4, wherein the surface-active agent is selected from the group of surfactants consisting of: cations, anions, amphoteric moieties, non-ionic or zwitterionic compounds, benzalkonium chloride, benzethonium chloride, methylbenzethonium chloride, cetalkonium chloride, cetylpyridinium chloride, cetrimonium, cetrimide, dofaniumchloride, tetraethylammonium bromide, domiphen bromide, phenols, phenolic derivatives, cresols, terpenes, terpenoids, phenylpropanoids and polychloropheoxy phenols, eugenol, thymol, eucalyptol, malaleuca, carvacrol or cinnamaldehyde, plant derived volatile oils, derivatives of plant derived volatile oils.

8. (canceled)

9. (canceled)

10. (canceled)

11. (canceled)

12. The formulation of claim 1, wherein the membrane permeabilizing entity is least one compound selected from the group consisting of: ethylenediaminetetra acetic acid, citric acid, gluconic acid, malonic acid, polyphosphates, lactoferrin, transferrin, lactoferricin B, chlorhexidine, alexidine, vantocil, and polyhexamethylene biguanides.

13. (canceled)

14. (canceled)

15. The composition of claim 1, wherein the membrane permeabilizing entity is included in an emulsion, a suspension as a particle in oil in water (o/w) emulsion, nanoparticle, or a micelle.

16. The formulation of claim 15, wherein the membrane permeabilizing entity is further activated to interact with a bacteria cell membrane by contact with bodily fluids.

17. (canceled)

18. (canceled)

19. (canceled)

20. (canceled)

21. (canceled)

22. (canceled)

23. The formulation of claim 1, wherein the composition includes at least one additional antimicrobial compound selected from the group consisting of: silver, selenium, and antibiotics.

24. (canceled)

25. The formulation of claim 23, wherein the additional antimicrobial compound acts synergistically with the hyperosmotic and/or membrane permeabilizing entity.

26. The formulation of claim 1, wherein the hyperosmotic component is selected from the groups consisting of: a saccharide component with an osmolarity between about 0.45 OsM to about 4.5 OsM; or a polyol component having an osmolarity between about 0.25 OsM to about 10.0 OsM; or a saccharide and polyol component having an osmolarity of between about 0.25 OsM to about 10.0 OsM.

27. The formulation of claim 1, wherein the wound dressing formulation has a water activity of between about 0.42 to about 0.99.

28. The formulation of claim 1, wherein the formulation is in an aqueous state, and concentration of the membrane permeabilizing entity in the formulation is between about 0.0001% to about 4.5% (w/w) of the formulation.

29. (canceled)

30. (canceled)

31. (canceled)

32. (canceled)

33. The formulation of claim 1, which further includes an absorbent agent.

34. The formulation of claim 33, wherein the absorbent agent is derived from at least one compounds selected from the groups consisting of: alginic acid or salt thereof; carrageenan; a derivative of alginic acid or salt thereof and a derivative of carrageenan.

35. The formulation of claim 1, wherein the formulation is in the form of a liquid, pliable solid, semi-solid paste, gel, expanding foam, hydrocolloid, ointment, or cream.

36. The formulation of claim 1, wherein the formulation is provided as a non-woven fibrous pad.

37. (canceled)

38. (canceled)

39. The formulation of claim 1, further including an agent that indicates the osmolarity of the dressing and, wherein the said indicator is a chemical that changes color in response to changes is osmolarity.

40. A method of reducing the number of microorganisms on a surface, comprising the steps of: contacting a surface with a formulation according to claim 1.

41. The method according to claim 40, wherein the surface is a site selected from the group of sites consisting of: an acute traumatic wound; a chronic non-healing wound, a recovering skin graft; a pressure ulcer; a surgical insult, a skin deformity; a cancerous lesion; an oral wound; acne, a viral infection; and a fungal infection.
